(19) United States
(12) Patent Application Publication
(10) Pub. No.: US 2015/0359418 A1
(43) Pub. Date: Dec. 17, 2015

(54) ENDOSCOPE, PARTICULARLY FOR MINIMALLY INVASIVE SURGERY

(71) Applicant: SIEMENS AKTIENGESELLSCHAFT, München (DE)

(72) Inventors: Hubertus FEUSSNER, Munich (DE); Peter RENTSCHLER, Neuhengstett (DE); Anton SCHICK, Velden (DE); Patrick WISSMAN, Munich (DE)

(73) Assignee: SIEMENS AKTIENGESELLSCHAFT, München (DE)

(21) Appl. No.: 14/762,161

(22) PCT Filed: Nov. 29, 2013
(86) PCT No.: PCT/EP2013/075042

§ 371 Date: Jul. 20, 2015

(30) Foreign Application Priority Data
Jan. 21, 2013 (DE) 102013200898.8

(51) Int. Cl. A61B 1/00; A61B 5/055; A61B 6/03; A61B 1/005

(52) U.S. Cl. CPC A61B 1/00193; A61B 1/00009; A61B 1/005; A61B 5/055; A61B 6/03

(57) ABSTRACT

Three-dimensional detection of an interior space of a body is performed by an endoscope in which a projection device projects a color pattern onto a region of the interior space and a detecting device detects an image of the color pattern projected onto the region. The projection and detection devices are positioned at least in part in a distal end region of an elongate endoscope extent. The distal end region can be angled up to 180° in relation to the original elongate endoscope extent. Active triangulation can be used in evaluating 3D images of the region to simply and effectively enlarge the 3D images. Such endoscopes can be used particularly advantageously in minimally invasive surgery or in industrial endoscopy.

### CROSS REFERENCE TO RELATED APPLICATIONS

This application is the U.S. national stage of International Application No. PCT/EP2013/075042, filed Nov. 29, 2013 and claims the benefit thereof. The International Application claims the benefit of German Application No. 102013200898.8 filed on Jan. 21, 2013, both applications are incorporated by reference herein in their entirety.

### BACKGROUND

Described below is an endoscope, particularly for minimally invasive surgery.

In comparison with frequently conventional open surgery, numerous methodological-technical restrictions apply to minimally invasive or laparoscopic and in particular scarless surgery. The restrictions relate primarily to visualization, spatial orientation, assessment of the tissue constitution and the spatial confinement of the work area with greatly reduced degrees of freedom. For this reason, complex interventions, in particular, have hitherto not yet been able to be carried out minimally invasively, even though this would be inherently very desirable.

Therefore, intensive research and development efforts are being made globally in order to extend the applicability of minimally invasive surgery.

One major disadvantage of conventional minimally invasive surgery is missing or inaccurate information about the third dimension, since only the organ surfaces are viewed and, for example, the sense of touch cannot be used to localize a tumor internally in an organ. The depth information could be conveyed, in principle, by the projection of volume data sets obtained preoperatively, but this form of augmented or enhanced reality conventionally fails for lack of reliable referencing. In comparison with preoperative diagnostics, intraoperatively a more or less pronounced position and shape change, for example of an intra-abdominal anatomy, can always occur, to which a preoperative data set has to be adapted in each case. Such an adaptation would be possible in terms of software if, in comparison with the related art, more exact information were available about a current surface of an organ for example in an abdominal space. In addition, conventionally a field of view is greatly restricted.

Numerous approaches propose a precise, continuous depth measurement in real time. Conventionally, it is not possible to determine accurate distances between a respective anatomy and the measurement objects used at every point in time of an intervention. The absence of this information is a cause of a large number of problems that currently still exist.

For the further development of medical operations via natural orifices of the body, a precise 3D metrology is the key technology. Without a successful implementation, NOTES (Natural Orifice Transluminal Endoscopic Surgery), or the minimally invasive surgery without scars, which involves operating by access through natural orifices of the body, will not be able to be introduced into clinical care. The use of mechatronic auxiliary systems is essential for NOTES. The systems in turn necessarily require a reliably functioning depth or 3D metrology for collision avoidance, for the compensation of breathing- or respiration-dictated organ deflections and a large number of further functions.

Various solution approaches used hitherto in other technical fields can be used to provide 3D information and corresponding 3D metrology.

### Stereoscopy

Stereoscopic triangulation is a known principle of distance measurement. In this case, an object is imaged from two observation directions by cameras. If a distinctive point is recognized in both recordings, then given a known distance between the cameras, the so-called base, a triangle is spanned which is uniquely determined with the base value and two angles and enables the distance of the point to be calculated. What is usually disadvantageous here, however, is the fact that there are too few distinctive points in the object and too few corresponding points are thus found in the cameras. Such a problem is referred to as the correspondence problem.

### Phase Triangulation

In order to avoid such a correspondence problem, so-called active triangulation has been used, which projects from one direction known patterns or, as in the case of phase triangulation, a sequence of sinusoidal patterns onto an object. As a result of the imaging of the object from another direction, the pattern appears distorted depending on the shape of the pattern surface, wherein the three-dimensional surface can in turn be calculated from this distortion, which is likewise referred to as a phase shift. This procedure enables even totally contrastless and markerless surfaces to be measured. What is disadvantageous about this type of 3D measurement in the field of minimally invasive surgery is an only minimal space for accommodating a camera and a projector—fitted at an angle—for projecting pattern sequences. A further disadvantage is that the position with respect to the object must not be altered during a projection sequence, since otherwise the 3D coordinate calculation is greatly beset by errors.

Time of flight

The disadvantage of the 3D coordinate calculation beset by errors on account of an object movement likewise occurs in the so-called time of flight (TOF) methods. Here, likewise, from a location of the object surface, at least four intensity values are measured for different times of flight of an intensity-modulated transmission signal. A computation of these intensity values produces a respective distance value. A further challenge however is in particular the measurement of the time-of-flight differences caused by distance differences in the millimeters range given the very high speed of light in the region of 300 000 km per second. Known systems can measure the distance of a single object point at a resolution of one millimeter by using highly developed detectors and electronics. Only inadequate values for surgery in the centimeters range are achieved for planar TOF distance sensors.

Structure from Motion

This method is based on the fact that, in principle, by the motion of a camera in front of an object, many images are recorded from different directions and triangulation is made possible again, in principle, in this way. However, the so-called correspondence problem arises again in this case, that is to say that a distinctive point has to be recognized in the respective sequential images. Furthermore, it is not possible to calculate absolute, but rather only relative values, since the triangulation base, the distance and the orientation between the temporal recordings are not known or would additionally have to be measured by tracking systems.

### SUMMARY

The problem addressed is that of providing an endoscope such that a visualization, a spatial orientation and/or an assessment of an object, in particular of tissue, in particular in the case of a spatial confinement of a work volume with reduced degrees of freedom, are/is improved and simplified in comparison with conventional systems. In particular, an applicability to minimally invasive surgery is intended to be extended. Complex minimally invasive interventions are likewise intended to be implementable. A precise, continuous depth measurement in real time is intended to be made possible and accurate distances between endoscope and object are intended to be determinable at every point in time of an intervention. An endoscopic apparatus is intended to be provided such that 3D measurement data of surfaces, in particular in the field of minimally invasive surgery, are generated with a higher data quality in comparison with the related art.

For the integration of optical systems particularly in the field of minimally invasive surgery (MIC), it is important that the optical systems are sufficiently miniaturizable and nevertheless do not lose their performance in the sense of imaging or measurement accuracy. It is necessary to overcome the disadvantage that a reduction of dimensions in an optical system generally likewise means a loss of information transmission capacity, be it that the size of a field of view is reduced or that the resolution capability is reduced. This concerns 3D metrology, in particular since the latter has to likewise transmit the third dimension.

In accordance with one aspect, an endoscope for three-dimensionally detecting a region of an internal space is proposed, wherein the endoscope extends along an original elongate endoscope extent as a longitudinal body having a distal end region which can be angled by up to 180°, in particular up to 110° or 90°, with respect to the original elongate endoscope extent, wherein an apparatus for three-dimensionally detecting the region by active triangulation is formed at least partly in the distal end region.

The three-dimensionally measuring optical system proposed makes it possible to produce measurements of distance to individual points of a surface of an internal space and more exact information about an internal space of a body. An endoscopic apparatus is proposed which, particularly for minimally invasive surgery, provides three-dimensional measurement data of surfaces with higher data quality in comparison with the related art. So-called active triangulation is particularly advantageously used, which projects from one direction known patterns or, as in the case of phase triangulation, a sequence of sinusoidal patterns onto an object. Configurations such as are known from DE 10 232 690 A1 are particularly advantageous.

In accordance with one advantageous configuration, the apparatus for three-dimensionally detecting the region can have a projection device for projecting an, in particular redundantly coded, color pattern onto the region and a detection device for detecting an image of the color pattern projected onto the region.

In accordance with a further advantageous configuration, a transmission device can be designed for transmitting the image generated by the detection device to an evaluation device for processing the image to form three-dimensional object coordinates which can be represented as a 3D image for an operator by a display device.

In accordance with a further advantageous configuration, the projection device and/or the detection device can be formed at least partly in the distal end region.

In accordance with a further advantageous configuration, the projection device and the detection device can be formed completely or one of the two can be formed completely and the other is formed partly in the distal end region in such a way that both have in each case a viewing direction substantially perpendicular to the elongate extent of the angled distal end region.

In accordance with a further advantageous configuration, the two viewing directions can be rotatable about a rotation axis running along the elongate extent of the distal end region, in particular an axis of symmetry of the distal end region. A restricted field of view can be extended in this way since, by a depth map, a large number of individual images of the internal space can be joined together to form a virtual panorama, which can likewise be referred to as “mosaicing” or “stitching”. Such an extension of the field of view can considerably facilitate performance of an operation, for example, and effectively improve a safety level.

In accordance with a further advantageous configuration, either the projection device or the detection device can be formed completely and the other is not formed in the distal end region and both can have substantially parallel viewing directions in an angled state.

In accordance with a further advantageous configuration, the two viewing directions substantially can run along the original elongate endoscope extent.

In accordance with a further advantageous configuration, it is possible that the endoscope can be angled by approximately 90° with respect to the regional elongate endoscope extent.

In accordance with a further advantageous configuration, a portion of the projection device and of the detection device which is not formed in the distal end region can be formed in the longitudinal body adjoining the distal end region.

In accordance with a further advantageous configuration, a portion of the projection device and of the detection device which is not formed in the distal end region can be formed outside the longitudinal body at a side of a proximal end region of the longitudinal body.

In accordance with a further advantageous configuration, the detection device or the projection device can be formed outside the longitudinal body and the other is formed in the distal end region.

In accordance with a further advantageous configuration, proceeding from the detection device or projection device formed outside the longitudinal body, an image guide device can be formed into the longitudinal body to an objective adjoining the distal end region in the distal end region.

In accordance with a further advantageous configuration, if the projection device is formed in the distal end region, a light guide device to the projection device can be formed from a light source outside the longitudinal body into the longitudinal body.

In accordance with a further advantageous configuration, it is possible that the endoscope can be rigid and the distal end region can be angled by a joint.

In accordance with a further advantageous configuration, it is possible that the endoscope can be flexible and the distal end region can be angled by a flexible material or a joint.

In accordance with a further advantageous configuration, the endoscope has a mechanical mechanism or electromechanical mechanism by which the distal end region can be angled.

In accordance with a further advantageous configuration, the transmission device can transmit the image by at least one transmission medium from the detection device to the evaluation device.

In accordance with a further advantageous configuration, optical or electrical image data can be detectable by mirrors, electrical lines, light guides or transparent or electrically conductive layers as transmission media.

In accordance with a further advantageous configuration, a position determining device can be formed, by which a position of the projection device and of the detection device can be determinable.

In accordance with a further advantageous configuration, the projection device can project white light onto the region of the internal space alternately to the color pattern, and the detection device can detect color images of the region alternately to 3D images which are calibratable by the white light.

In accordance with a further advantageous configuration, the display device can provide the 3D images and the color images of the region in real time for an operator.

In accordance with a further advantageous configuration, the detection data rate of the 3D images and of the color images can be in each case between 20 and 40 Hz, in particular 25 Hz.

In accordance with a further advantageous configuration, the evaluation device can fuse three-dimensional object coordinate data of the region with point cloud data of the region obtained by at least one further measuring device, in particular a magnetic resonance imaging device or a computed tomography device.

### BRIEF DESCRIPTION OF THE DRAWINGS

These and other aspects and advantages will become more apparent and more readily appreciated from the following description of exemplary embodiments, taken in conjunction with the accompanying drawings of which:

FIG. 1A is a schematic cross section of a first exemplary embodiment of an endoscope in a first operating mode;

FIG. 1B is a schematic cross section of the first exemplary embodiment of an endoscope in a second operating mode;

FIG. 1C is a perspective view of an exemplary embodiment of a known endoscope;

FIG. 2 is a schematic cross section of a second exemplary embodiment of an endoscope;

FIG. 3 is a schematic cross section and block diagram of a third exemplary embodiment of an endoscope;

FIG. 4A is a schematic cross section of a fourth exemplary embodiment of an endoscope in a first operating mode;

FIG. 4B is a schematic cross section of the fourth exemplary embodiment of an endoscope in a second operating mode;

FIG. 5 is a schematic cross section of a fifth exemplary embodiment of an endoscope;

FIG. 6 is a schematic cross section of a sixth exemplary embodiment of an endoscope;

FIG. 7 is a perspective view of an exemplary embodiment of a known position determining apparatus;

FIG. 8A is a side view of an exemplary embodiment of an endoscope in an internal space at a first point in time;

FIG. 8B is a side view of the exemplary embodiment of an endoscope in accordance with FIG. 8A at a second point in time.

### DETAILED DESCRIPTION OF THE PREFERRED EMBODIMENT

Reference will now be made in detail to the preferred embodiments, examples of which are illustrated in the accompanying drawings, wherein like reference numerals refer to like elements throughout.

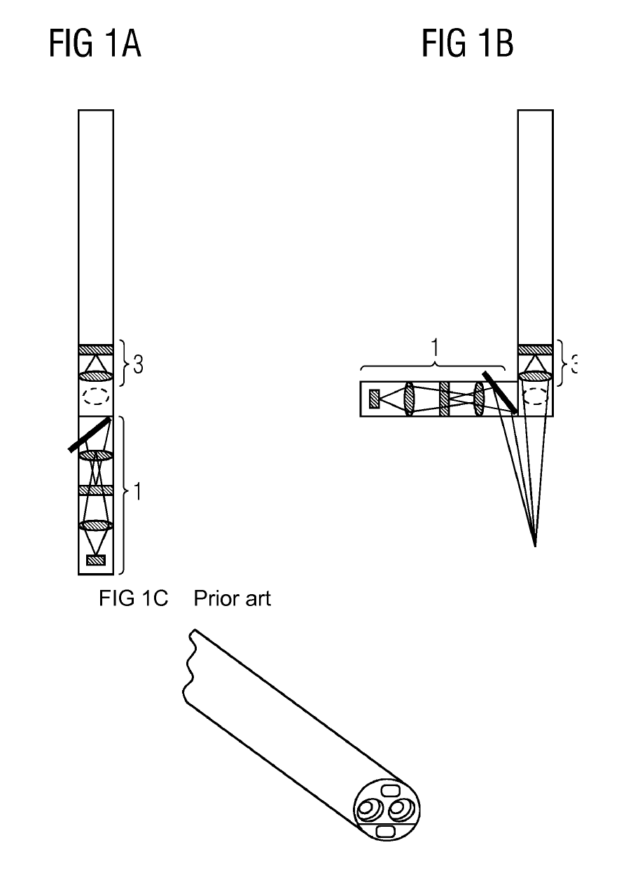

FIG. 1A shows a first exemplary embodiment of an endoscope in a first operating mode, in which the endoscope can be inserted into an abdominal space for example through a trocar. The illustrated endoscope for three-dimensionally detecting an internal space is in an initial state in which a longitudinal body having a distal end region extends along an original endoscope extent without being angled. In accordance with this exemplary embodiment, a projection device 1, for example a projector, in particular a slide projector, for projecting a color pattern, in particular a singly or redundantly coded color pattern, onto an object is arranged in the distal end region of the longitudinal body.

The projection device 1 here is positioned completely in the distal end region. Further component parts of a projection device 1 can be a light source, for example at least one light emitting diode LED, drive electronics and further known projector elements. A detection device 3, for example a camera, for detecting an image of the color pattern projected onto the object is arranged outside the distal end region in the longitudinal body adjoining the distal end region. In accordance with the exemplary embodiment in accordance with FIG. 1A, the detection device 3 and projection device 1 are positioned one behind the other in this order in the direction of a distal end of the endoscope. The distal end region can be angled with respect to the original elongate endoscope extent here by up to 90°. Instances of angling by up to 180° or for example by 110°, are likewise possible, in principle. In accordance with this exemplary embodiment, the projection device 1 is arranged in the bendable part of the endoscope. The detection device 3 is arranged with a viewing direction along the original elongate endoscope extent in the non-bendable part of the endoscope. The distal end region is embodied such that it can be angled partly with respect to the original elongate endoscope extent in such a way that the projection device 1 can be angled with respect to the original elongate endoscope extent. In all embodiments, a transmission device (not illustrated) is provided, by which, in particular, image data or images from the detection device 3 can be transmitted to an evaluation device 7 (not illustrated here). In principle, data transmission to and from the projection device 1 and the detection device 3 can be provided in all embodiments. Driving and reading of the projection device 1 and of the detection device 3 can be implemented in this way.

FIG. 1B shows the first exemplary embodiment of an endoscope in a second operating mode, in which three-dimensional data can be obtained. In this case, the projector is situated in the angled region and the camera is situated in the long shaft or non-angled part of the longitudinal body of the endoscope. The distal end region has been angled at 90° with respect to the original elongate endoscope extent in such a way that the projection device 1 has likewise been angled by 90° with respect to the original elongate endoscope extent. In accordance with this operation mode, the projection device 1 and the detection device 3 in each case have a viewing direction substantially along the original elongate endoscope extent, in the case of a corresponding orientation in particular in the direction of an object, for example a surface of an internal space. It is particularly advantageous if the endoscope latches after being angled and is mechanically fixed or held in this way. An endoscopic apparatus that provides three-dimensional measurement data of surfaces with a high data quality is provided in this way. This is brought about by the endoscope being mechanically bendable at a defined location. A relatively large triangulation base in comparison with the related art for the active triangulation used and thus a high depth resolution are brought about in this way. By way of example, it is possible to provide a depth resolution of 0.5 mm at a distance of 10 cm. What is advantageous in the embodiments is that the triangulation base can be disposed as a measure of an achievable depth resolution with an order of magnitude of 2-4 cm. In comparison with conventional endoscopes, a depth resolution can be increased by approximately the factor of 10 in the case of the endoscopes described herein.

FIG. 1C shows an exemplary embodiment of a known endoscope. In the case of such a known endoscope, both projector and camera optical unit are arranged at a front distal end face and have a viewing direction toward the front. Given a typical diameter of such an endoscope in the region of approximately 10 mm, the triangulation base is thus in the range of approximately 3-4 mm.

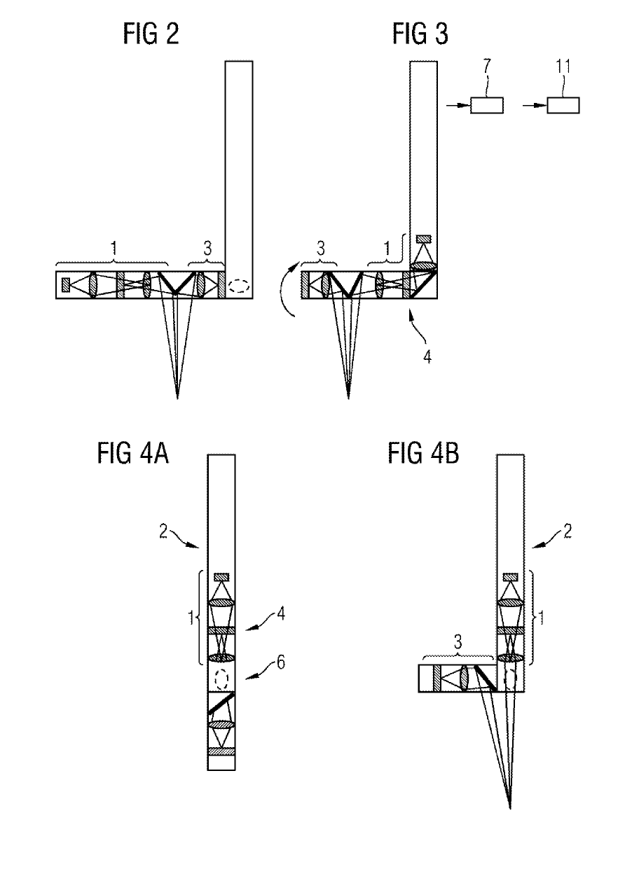

FIG. 2 shows a second exemplary embodiment of an endoscope. In accordance with this embodiment, a projection device 1 and a detection device 3 are arranged completely in the distal end region and are angled by 90° with respect to the original elongate endoscope extent. In accordance with FIG. 2, the projection device 1 is arranged at the distal end of the endoscope. The detection device 3 is positioned nearer to the proximal end of the endoscope alongside the projection device 1 in the distal end region. In the angled operating mode illustrated here, the projection device 1 and the detection device 3 in each case have a viewing direction substantially perpendicular to the elongate extent of the distal end region. In accordance with FIG. 2, a projector and a camera are arranged in the bendable part of the endoscope. A joint, for example, is arranged at the mechanically bendable location, wherein it is possible to deflect optical and electrical signals from the distal end region, by mirrors, wires, lights or transparent, electrically conductive layers. In accordance with FIG. 2, a projector and a receiver, which is embodied as a camera, are arranged in the bendable part of the endoscope or in the angled distal end region. A combination with a deflection device for deflecting optical and electrical signals is additionally possible, wherein a deflection can be intimated here by elements of the detection device 3. In the case of an interchanged arrangement, the deflection can be brought about by elements of the positioning device.

FIG. 3 shows a third exemplary embodiment of an endoscope. In accordance with this embodiment, a detection device 3 is arranged completely and a projection device 1 is arranged partly in the distal end region that can be angled. A portion of the projection device 1 that is not formed in the distal end region is formed in the longitudinal body adjoining the distal end region. For this purpose, by way of example, a camera can be formed in the angled region and a projector can be formed partly in the angled region and partly in a rigid shaft. A transparency 4, for example, can be arranged in the transition region from the region that cannot be angled to the region that can be angled. As in all the embodiments, a transmission device (not illustrated) is provided, by which, in particular, image data from the detection device 3 can be transmitted to an evaluation device 7. In principle, in all the embodiments, data transmission into and out of the distal end region or the angled distal end region and also to and from the projection device 1 and the detection device 3 can be provided or is provided.

In accordance with FIG. 3, the projection device 1 and detection device 3 in each case have a viewing direction substantially perpendicular to the elongate extent of the distal end region. FIG. 3 shows with an arrow on the left of the detection device 3 that the two viewing directions of the projection device 1 and of the detection device 3 are rotatable about a rotation axis running along the elongate extent of the distal end region, in particular an axis of symmetry of the distal end region. A field of view of the endoscope can be effectively extended in this way. A panoramic image, for example, can be generated by a plurality of individual images being joined together. In accordance with FIG. 3, a projection device 1 is formed partly in the distal end region that can be angled. In this case, part of the projection device 1 remains in the region of the endoscope that cannot be angled. In accordance with FIG. 3, the bendable distal end region is rotatable together with the field of view of a projector and the field of view of a camera about a cylinder axis of the distal end region, such that data fusion and an enlargement of a field of view are made possible by progressive measurement in the case of overlapping measurement fields or measurement regions of an endoscope.

FIG. 4A shows a fourth exemplary embodiment of an endoscope in a first operating mode, which is used for example for inserting the endoscope into an abdominal space or a technical internal space. FIG. 4a shows a projector or a projection device 1 in a rigid part of an endoscope, wherein this proximal region can be referred to as endoscope shaft. Proximal means the side which is nearer to the operator. A distal side means the side that is formed further away from an operator. The projector can have a transparency 4; the endoscope shaft bears the reference sign 2. FIG. 4a shows an endoscope in a first operating state, in which no angling was carried out. Bending can be made possible by a joint 6.

FIG. 4B shows the fourth exemplary embodiment of an endoscope in a second operating state. For this purpose, a camera as detection device 3 is positioned in the distal end region that can be angled, and is rotated out of the position in the first operating state or initial state here by 90°. The bending is made possible here by a joint 6. Other configurations are likewise possible, in principle. In FIG. 4b, projector has a viewing direction downward. In FIG. 4b, the camera or detection device 3 is likewise formed with a viewing direction downward in the bendable part of the endoscope.

FIG. 5 shows a fifth exemplary embodiment of an endoscope. A detection device 3 is formed outside the longitudinal body and a projection device 1 is formed in the distal end region. Therefore, a portion of the projection device 1 and of the detection device 3 which is not formed in the distal end region is formed outside the longitudinal body at a side of a proximal end region of the longitudinal body. Proceeding from the detection device 3, an image guide device 13 is formed from outside the longitudinal body in the longitudinal body to an objective 15 adjoining the distal end region in the longitudinal body.

Using a light guide, an image of an object can thus be detected by the detection device 3 by the objective 15. In accordance with FIG. 5, the projection device 1 is formed in the distal end region and receives from a light source 17 outside the longitudinal body, by a light guide device 19, light for projecting color patterns and/or for illuminating an object with white light. Since the light source 17 is external, it can provide a high light power. Heat losses can simply be dissipated. The projection device 1 here can be formed completely in the distal end region.

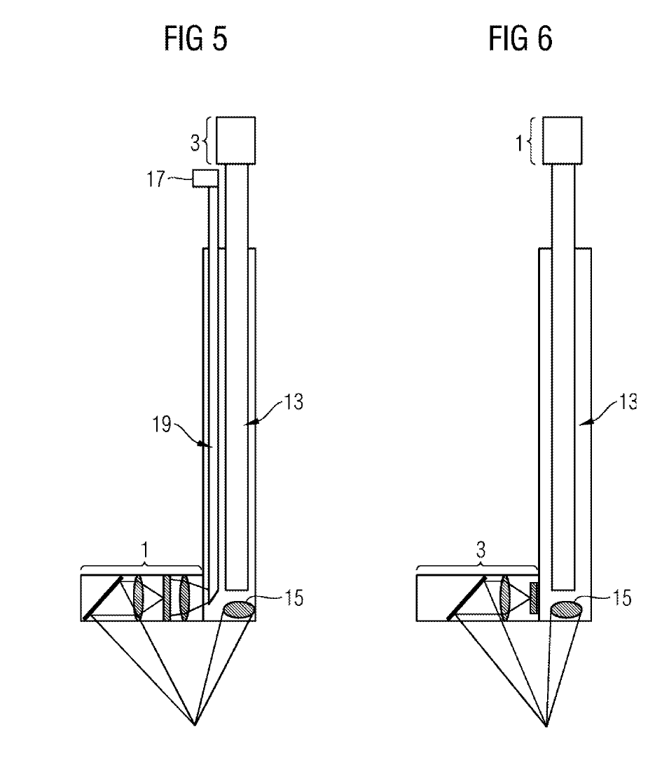

FIG. 6 shows a sixth exemplary embodiment of an endoscope. A projection device 1 is formed outside the longitudinal body and a detection device 3 is formed in the distal end region. Therefore, a portion of the projection device 1 and of the detection device 3 which is not formed in the distal end region is formed outside the longitudinal body at a side of a proximal end region of the longitudinal body. Proceeding from the projection device 1, an image guide device 13 is formed from outside the longitudinal body in the longitudinal body to an objective 15 adjoining the distal end region in the longitudinal body. Using a light guide, a color pattern can be projected onto an object by the objective 15. The detection device 3 here is formed completely in the distal end region.

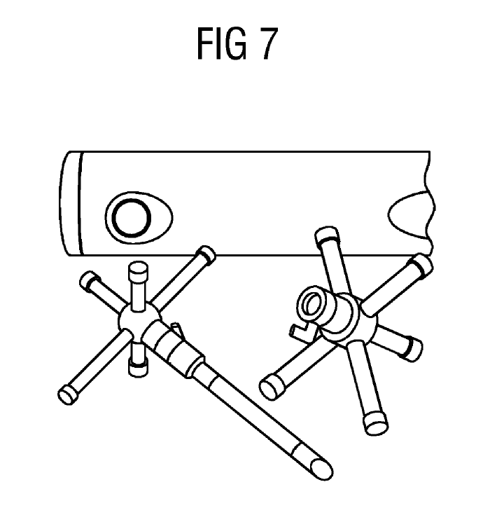

FIG. 7 shows an exemplary embodiment of a known position determining apparatus which can supplement an endoscope. If an endoscope is formed with a position determining apparatus, which can likewise be referred to as a tracking apparatus, a measured and detected surface of an operation site, for example, can be linked with the endoscope position obtained. FIG. 7 shows a known embodiment using electromagnetic or optical tracking. Further alternatives include fitting distinctive structures, for example spheres, in an outer region of the endoscope or tracking by optical triangulation. Further position determining apparatuses are likewise possible.

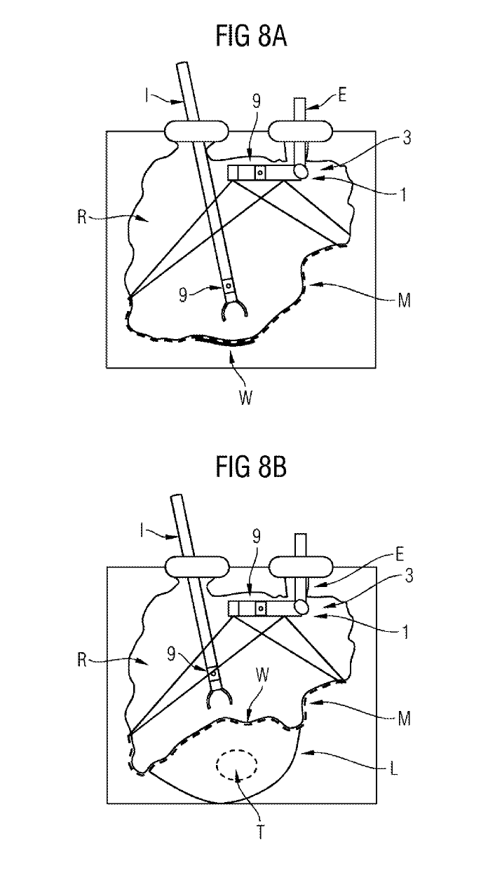

FIG. 8A shows an exemplary embodiment of an endoscope in an internal space at a first point in time. In this case, in accordance with this exemplary embodiment, the endoscope is optimally adapted to the band recognitions of minimally invasive surgery. For this purpose, the endoscope E is formed as a measured endoscope and is insertable and here inserted into an air-filled abdominal space, as an example of internal space, through a trocar. The insertion took place from above here, the intention being to carry out an operation on a liver L. The endoscope E is deflected by approximately 90° at a defined bend here at the first point in time, such that the viewing direction of a projection device 1 in the form of a projector and of a detection device 3 in the form of an imaging optical unit here is directed downward at the operation region in the interior of the abdominal space.

The endoscope E enables an enlargement of a triangulation base and measurements of surfaces and the 3D extents thereof in real time. Thus, it is now possible to enlarge a usable cross-sectional area for the optical components of the endoscope E. The Lagrange invariant can be increased, this being a measure of the optical information transmission performance in optics. In this way, an effectively higher lateral resolution and a depth resolution are brought about particularly in the 3D area in the endoscope. Equally, in comparison with the related art in accordance with FIG. 1C, it is possible to effectively enlarge the cross-sectional area for supplying light, which corresponds to an increase in the etendue. The measurement surfaces detectable in real time are identified by M in FIGS. 8A and 8B. A position determining device 9 advantageously detects the position of the projection device 1 and of the detection device 3 and also, in particular, the position of the triangulation base and makes it possible in this way likewise to determine the positions of the detected surface structures relative to an external coordinate system. A further position determining device 9 can be arranged on an additional instrument I, such that the position thereof can likewise be determined with respect to the external coordinate system. It is thereby possible to localize the measuring system relative to the instrument. In this way, an operator can be supplied with additional information for operation within an internal space. Reference sign W identifies a region to be treated or processed in the internal space in which the endoscope E and instrument I have been introduced. A transmission device (not illustrated here) transmits the image generated by the detection device 3 to an external evaluation device 7 for processing the image to form three-dimensional object coordinates. Using a display device 11 (not illustrated here), an operator can see a 3D image of a region W of the internal space. The projection device 1 can project white light onto the region W of the internal space alternately to the color pattern, and the detection device 3 can detect color images of the region W alternately to 3D images which are calibratable by the white light. In this way, in addition to the 3D images, the display device 11 can provide color images of the region W in real time for an operator. In the case of such alternate image recording with structured illumination and illumination with white light it is possible to calculate depth data, when the light white recording in this case can serve for color correction of color fringes and a disturbing influence of the color of the object or of the region W can be reduced in this way. The alternate image recording with structured illumination and illumination with white light likewise makes it possible to visualize a region W to be processed, for example an operation site for a surgeon, by a display of a color image. At an image rate of 50 hertz, the surface of an operation seen or of a 3D surface region W can be calculated in real time—for example at 25 Hz—and can be used as a data set for navigation, specifically guiding the surgeon to the disease center or the operator to the site of use, and are represented on the display device 11 for the operator. At the same time, the color image can be displayed in real time—for example at an image rate of 25 Hz—for the purpose of orientation for the operator or the surgeon in the site of use or abdominal space for example on a monitor or a head-up display. Furthermore, information for the navigation or guiding can be inserted on a or the monitor, for example arrows.

FIG. 8B shows the exemplary embodiment of an endoscope in accordance with FIG. 8A during a second point in time. Reference signs identical to those in FIG. 8A identify identical elements. In accordance with FIG. 8B, an embodiment of an endoscope E can be used in which the projection device 1 can project white light on the region W of the internal space alternately with respect to the coded color pattern and the detection device 3 can detect color image data of the region W alternately with respect to calibratable 3D image data.

FIG. 8b shows the second point in time, at which the operator, specifically here a surgeon, uses point cloud data of the region W obtained by at least one further measuring device, in particular a magnetic resonance imaging device or a computed tomography device, in addition to images and 3D images. In this case, the evaluation device 7 can fuse three-dimensional object coordinate data of the region W or a 3D image with a point cloud data of the region that are obtained by at least one further measuring device, in particular a magnetic resonance imaging device or a computed tomography device. Using this additional information, the region to be treated, for example a liver L, can be detected by the detection device 3 in such a way that defective locations or diseased tissue, for example a tumor T, can be localized and removed. When a 3D endoscope is used as measuring means for the three-dimensional measurement of a surface of an organ, the fusion with in particular preoperatively obtained point clouds is additionally performed in accordance with FIG. 8B. Such point clouds may have been provided for example by nuclear spin tomography device or a magnetic resonance imaging device. In this case, a preoperatively obtained surface of an organ is determined in a point cloud and is deformed in a data set in such a way that the point cloud has the form of the surface form measured by an endoscope E. In this case, the points of the point cloud are elastically linked with one another, such that regions within an organ correspondingly concomitantly deform during a surface deformation and, if appropriate, adopt a new position. If, for example, the tumor T is situated within an organ, for example the liver L, and if the tumor T is localizable in the preoperatively obtained point cloud, then a change in the position of the tumor T can be determined by the 3D/3D data fusion and used as information for the navigation of the surgeon to the disease center. The endoscopes are particularly advantageous high-resolution 3D endoscopes in particular for minimally invasive surgery. In principle, the endoscopes are not restricted to medical applications. Further areas of application are found in technical endoscopy or wherever internal spaces have to be detected, tested, monitored or processed.

An endoscope for three-dimensionally detecting an internal space R of a body are disposed, wherein a projection device 1 for projecting a color pattern onto a region W of the internal space R and a detection device 3 for detecting an image of the color pattern projected onto the region W are positioned at least partly in a distal end region of an elongate endoscope extent and the distal end region can be angled by up to 180° with respect to the original elongate endoscope extent. A triangulation base for evaluating images by active triangulation for generating 3D images of the region W can be simply and effectively enlarged in this way. Such endoscopes can particularly advantageously be employed in minimally invasive surgery or in technical endoscopy.

A description has been provided with particular reference to preferred embodiments thereof and examples, but it will be understood that variations and modifications can be effected within the spirit and scope of the claims which may include the phrase “at least one of A, B and C” as an alternative expression that means one or more of A, B and C may be used, contrary to the holding in Superguide v. DIRECTV, 358 F3d 870, 69 USPQ2d 1865 (Fed. Cir. 2004).

1-16. (canceled)

17. An endoscope for three-dimensionally detecting a region of an internal space, the endoscope, comprising:
a longitudinal body, extending along an original elongate endoscope extent, having a distal end region capable of being angled by up to 180° with respect to the original elongate endoscope extent of the longitudinal body;
an apparatus, detecting the region of the internal space in three-dimensions using active triangulation, formed at least partly in the distal end region, the apparatus including
a projection device projecting a coded, color pattern onto the region, and
a detection device detecting an image of the color pattern projected onto the region, at least one of the projection device and the detection device formed completely in the distal end region and both formed at least partly in the distal end region, each of the projection device and the detection device having a respective viewing direction substantially perpendicular to an angled elongate extent of the angled distal end region.

18. The endoscope as claimed in claim 17, wherein the respective viewing direction of each of the projection device and the detection device is rotatable about an axis of rotation, in particular about an axis of symmetry, along the angled elongate extent of the distal end region.

19. The endoscope as claimed in claim 17, connected to an evaluation device for processing the image to form three-dimensional object coordinates which can be represented as a three-dimensional image for an operator by a display device, and further comprising a transmission device transmitting the image generated by the detection device to the evaluation device.

20. The endoscope as claimed in 19, wherein the transmission device transmits the image by at least one transmission medium from the detection device to the evaluation device.

21. The endoscope as claimed in claim 20, wherein at least one of optical and electrical image data are detectable after transmission by at least one of mirrors, electrical lines, light guides, and transparent, electrically conductive layers.

22. The endoscope as claimed in claim 19, wherein the evaluation device receives point cloud data of the region from at least one of a magnetic resonance imaging device and a computed tomography device and fuses three-dimensional object coordinate data of the region with the point cloud data.

23. The endoscope as claimed in claim 17, wherein the endoscope can be angled by approximately 90° with respect to the original elongate endoscope extent.

24. The endoscope as claimed in claim 17, wherein a portion of at least one of the projection device and the detection device not formed in the distal end region is formed in the longitudinal body adjoining the distal end region.

25. The endoscope as claimed in claim 17, further comprising a side portion formed outside the longitudinal body at a side of a proximal end region of the longitudinal body in which a portion of at least one of the projection device and the detection device not formed in the distal end region is formed.

26. The endoscope as claimed in claim 25,
wherein the projection device is formed in the distal end region, and
wherein the endoscope further comprises:
a light source outside the longitudinal body, and
a light guide device formed from the light source to the projection device.

27. The endoscope as claimed in claim 17,
wherein the endoscope is rigid, and
wherein the endoscope further comprises a joint enabling the distal end region to be angled.

28. The endoscope as claimed in claim 17,
wherein the endoscope is flexible, and
wherein the endoscope further comprises at least one of a flexible material and a joint enabling the distal end region to be angled.

29. The endoscope as claimed in claim 17, further comprising at least one of a mechanical mechanism and a electromechanical mechanism by which the distal end region can be angled.

30. The endoscope as claimed in claim 17, further comprising a position determining device, by which a position of each of the projection device and the detection device is determinable.

31. The endoscope as claimed in claim 17,
wherein the projection device projects white light onto the region of the internal space alternately to the color pattern, and
wherein the detection device detects color images of the region alternately to three-dimensional images which are calibratable by the white light.

32. The endoscope as claimed in claim 31, wherein the endoscope is connected to a display device providing the three-dimensional images and the color images of the region in real time for an operator.

33. The endoscope as claimed in claim 32, wherein a detection data rate of the three-dimensional images and the color images is in each case between 20 and 40 Hz.

34. The endoscope as claimed in claim 33, wherein a detection data rate of the three-dimensional images and the color images in each case is substantially 25 Hz.
